(19) United States
(12) Patent Application Publication
(10) Pub. No.: US 2020/0329982 A1
(43) Pub. Date: Oct. 22, 2020

(54) AUTOMATED DETECTION AND RECOGNITION OF ADVERSE EVENTS

(71) Applicant: Bayer Aktiengesellschaft, Leverkusen (DE)

(72) Inventors: Ulf HENGSTMANN, Overath (DE); Christian Johannes MÜLLER, Düsseldorf (DE); Georg BERNS, Düsseldorf (DE); Sabine GENT, Bochum (DE)

(73) Assignee: Bayer Aktiengesellschaft, Leverkusen (DE)

(21) Appl. No.: 16/765,401

(22) PCT Filed: Nov. 19, 2018
(86) PCT No.: PCT/EP2018/081721

§ 371 Date: May 19, 2020

(30) Foreign Application Priority Data
Nov. 21, 2017 (EP) 17202790.6

(51) Int. Cl. A61B 5/0205; A61B 5/0464; A61B 5/00; A61B 5/11

(52) U.S. Cl. CPC A61B 5/02055; A61B 5/0464; A61B 5/7264; A61B 5/7275; A61B 5/6846; A61B 5/1118; A61B 5/0024; A61B 5/6801; A61B 5/74; A61B 5/4848

(57) ABSTRACT

A system, a method and a computer program product for automated detection and recognition of adverse events for monitoring the state of health of an individual

### CROSS REFERENCE TO RELATED APPLICATIONS

This application is a national stage application under 35 U.S.C. § 371 of International Application No. PCT/EP2018/081721, filed internationally on Nov. 19, 2018, which claims the benefit of European Application No. 17202790.6, filed Nov. 21, 2017.

### FIELD OF THE INVENTION

The present invention concerns the technical field of monitoring the health status of a person as part of a clinical or noninterventional study or as part of a therapy. The present invention relates to a system, a method and a computer program product for automatically detecting, capturing and processing so-called adverse events.

### BACKGROUND OF THE INVENTION

An adverse event (AE) is an unwanted incident which occurs as part of a clinical or noninterventional study or as part of a therapy in relation to a drug in a patient or a test person. In a clinical study, all adverse events must be carefully documented in case report forms provided for this purpose. At the same time, a trial physician has to provide an assessment as to whether he/she considers a causal link with the ingestion of the trial preparation to be possible. A case report form, CRF for short, is a data entry form for clinical or noninterventional studies, in which the visit data relating to a patient are documented by the physician in line with the trial plan or observation plan. This reporting is normally done in anonymized form. So-called “adverse events” recorded in said case report form can, in the event of a later authorization of a drug, be listed as side effects in the package insert. The case report form can be paper-based or be managed electronically as an eCRF.

Bradycardia and tachycardia are examples of adverse events. In medicine, bradycardia refers to a heart rate below 60 beats per minute in adult humans. A tachycardia is a persistent heart rate of above 100 beats per minute in adult humans; a pronounced tachycardia is referred to from a rate of 150 beats/min. Heart rate is influenced by various factors; it is generally known that heart rate increases under normal conditions in the event of physical stress; mental strain can bring about an increase in heart rate owing to shifting of the autonomic balance toward sympathetic activation; some stimulants as well, especially coffee, can make the heart beat faster.

### SUMMARY OF THE INVENTION

To identify the cause of an acute bradycardia or tachycardia, it is therefore necessary to observe the concomitant circumstances. In clinical studies or along with a therapy, use is increasingly being made of sensors which continuously and automatically capture features of the patients or test persons. The automatic monitoring of heart rate is nowadays a routine function in, for example, fitness trackers and smart watches. If an appliance for automatically monitoring heart rate is used in a study or as part of a therapy, what can occur is a multiplicity of events in which the heart rate is too high or too low, meaning the presence of signs of an adverse event. Each of these events must be checked in order to identify the adverse events which are attributable to the drug.

Thus, the permanent (i.e., 24 hours a day) and automatic capturing of physiological features has, on the one hand, the advantage of complete monitoring of the health status of a person, but, on the other hand, the disadvantage that a multiplicity of events requiring checking can occur.

According to some embodiments, the present invention provides a system for automatically detecting, capturing and processing adverse events as part of a clinical or noninterventional study or a therapy, comprising

a sensor,

a monitoring unit,

a classification unit,

a transmission unit,

a control unit,

the sensor being configured such that it automatically captures measurement values of one or more physiological parameters in a person who is being subjected to a medical treatment,

the monitoring unit being configured such that it analyzes the measurement values and identifies deviations from defined target values in the measurement values,

the classification unit being configured such that it analyzes the measurement values having deviations from defined target values and further personal data and/or environmental data and performs a classification into one of three classes,

class A: the deviations from defined target values are not a result of the medical treatment

class B: the deviations from defined target values are a result of the medical treatment

class C: a clear statement about the cause of the deviations from defined target values cannot be made

the control unit being configured such that it,

in the case of class A and class B, prompts the transmission unit to transmit a message about the presence of an adverse event to a computer system for capturing adverse events,

in the case of C, prompts the transmission unit to transmit measurement values to an expert for further clarification.

According to some embodiments, the present invention provides a method for automatically detecting, capturing and processing adverse events as part of a clinical or noninterventional study or a therapy, comprising the steps of:

capturing measurement values of one or more physiological parameters in a person who is being subjected to a medical treatment with the aid of a sensor,

analyzing the measurement values and identifying deviations from defined target values in the measurement values within an observation period,

ascertaining further personal data and/or environmental data,

classifying the observation period on the basis of the deviations from defined target values and the personal data and/or environmental data into one of three classes:

class A: the deviations from defined target values are not a result of the medical treatment

class B: the deviations from defined target values are a result of the medical treatment

class C: a clear statement about the cause of the deviations from defined target values cannot be made

transmitting a message about the presence of an adverse event to a computer system for capturing adverse events in the case of class A and class B or transmitting measurement values to an expert for further clarification in the case of class C.

According to some embodiments, the present invention provides a computer program product comprising a data carrier, and program code which has been saved on the data carrier and which prompts a computer, in the memory of which the program code has been loaded, to execute the following steps:

receiving measurement values of one or more physiological parameters of a person who is being subjected to a medical treatment as part of a clinical study, a noninterventional study or a therapy from a sensor,

analyzing the measurement values and identifying deviations from defined target values in the measurement values within an observation period,

classifying the observation period on the basis of the deviations from defined target values and the personal data and/or environmental data into one of three classes:

class A: the deviations from defined target values are not a result of the medical treatment

class B: the deviations from defined target values are a result of the medical treatment

class C: a clear statement about the cause of the deviations from defined target values cannot be made

transmitting a message about the presence of an adverse event to a computer system for capturing adverse events in the case of a classified sign of class A and B or

transmitting measurement values to an expert for further clarification in the case of a classified sign of class C.

The following descriptions are intended to apply analogously to all subjects of the invention, irrespective of the context (method, system, computer program product) in which they occur.

If steps are stated in an order in the present description or in the claims, this does not absolutely mean that the invention is limited to the order stated. According to some embodiments, the steps can be carried out in a different order or else in parallel to one another, unless one step builds on another step, this making it absolutely necessary for the building step to be carried out subsequently (this being clear, however, in individual cases). The orders stated are thus preferred embodiments of the invention of the method.

According to some embodiments, the present invention utilizes one or more sensors in order to monitor one or more physiological parameters in a person who is being subjected to a medical treatment.

Even though the present description uses the term “person”, this does not mean that the present invention is limited only to humans. It is also conceivable to monitor the health status of a different living organism, for example an animal, and to automatically detect, capture and process adverse events. For simplification, this description uses the terms “person”, “test person” and “patient”, which are also intended to encompass nonhuman living organisms. However, in a preferred embodiment, a human is concerned.

A “medical treatment” is understood to mean measures which have an influence on the health status of the person who is being subjected to the measure. The medical treatment can, for example, be a therapy, a clinical study or a noninterventional study.

A “therapy” refers to measures for treating diseases and injuries on the basis of a previously obtained diagnosis.

The term “clinical study” is understood to mean the experimental trial of a medical treatment method under defined basic conditions. It is carried out with patients or healthy test persons and is, for example, a prerequisite for official drug authorization. The German Arzneimittelgesetz [drugs act] defines a clinical trial as an “investigation carried out on humans that is intended to research or demonstrate clinical or pharmacological effects of drugs or to establish side effects or to study absorption, distribution, metabolism or excretion, with the goal of being assured of the harmlessness or efficacy of the drugs”. The goal of a clinical trial is to test medicaments, particular treatment forms, medical interventions or medical devices for their efficacy and safety.

In medical research, “noninterventional” studies refer to pure observational studies. Correspondingly, “noninterventional” means, for instance, “without any intervention during what is taking its course”. No medicaments, or medical devices, appliances or methods, are used, or only those which are authorized are used, in accordance with the particulars specified in the authorization. The patient is therapied as part of his/her routine treatment. The study does not provide the physician with any instructions in the form of a predetermined trial plan for treating the patient. The diagnosis methods and other observational methods are in line with medical practice.

Preferably, measurement values are captured by means of one or more sensors as part of a clinical or noninterventional study or as part of a therapy.

Preferably, a drug or a medical device is the subject of the clinical or noninterventional trial or the therapy.

“Drugs” are substances or preparations composed of substances that are intended for use in or on the human or animal body and are intended as agents having properties for healing or alleviating or for preventing human or animal diseases or pathological complaints, or that can be used in or on the human or animal body or administered to a human or an animal in order either to restore, correct or influence physiological functions through a pharmacological, immunological or metabolic effect or to make a medical diagnosis.

A term synonymous with the term drug is the term medicament. In this description, the terms “drug” and “medicament” are intended to also encompass trial preparations, for which there is yet no official drug authorization.

“Medical devices” are all instruments, apparatuses, devices, software, substances and preparations composed of substances or other objects that are used individually or in an interlinked manner which serve for the detection, prevention, monitoring, treatment or alleviation of diseases, for the detection, monitoring, treatment, alleviation or compensation of injuries or disabilities, for the study, replacement or modification of anatomical structure or a physiological process or for birth control and the intended main effect of which in or on the human body is achieved neither by pharmacologically or immunologically acting agents nor by metabolism, but the mode of action of which can be assisted by such agents.

According to some embodiments of the invention, one or more physiological parameters are automatically monitored with the aid of one or more sensors.

The term of “physiological parameters” is understood to mean a measurable variable providing information about the physical and biochemical state and/or the physical and biochemical processes in the cells, tissues and organs of a living organism. Examples of physiological parameters are: body weight, body temperature, heart rate, heart rhythm, (arterial) blood pressure, skin conductance, tremor (frequency), electrolyte/protein concentration or composition in body fluids, standard laboratory parameters, visual acuity, activity of specific brain areas, electrical activities of cardiac muscle fibers (e.g., captured by means of an electrocardiograph), central venous pressure, arterial oxygen saturation, respiratory rate—to name but a few. The effects caused in the interplay with drugs or by drugs in the body of a patient (pharmacokinetics, pharmacodynamics) and the effects of medical devices on the body of a patient are intended to be covered by the term of physiological parameters, too.

In a preferred embodiment, the physiological parameter is heart rate.

“Monitoring” means that measurement values are automatically captured and analyzed.

A “sensor” is a technical component which can capture certain physical or chemical properties and/or the material nature of its environment in a qualitative manner or in a quantitative manner as measurement variables. Said variables are captured by means of physical or chemical effects and converted into further-processable signals, usually electrical or optical signals.

Preferably, the sensor is one which is worn on a person's body (e.g., as a so-called “wearable”) or in a person's body (e.g., as a so-called “implantable”) continuously (at least during the observation period, which is usually at least one day, preferably at least one week). The sensor is thus preferably usable in a transportable and mobile manner. However, it is also conceivable that the sensor is not continuously carried by the person to be monitored, but is instead utilized at defined times by the person to be monitored. For example, it is conceivable that the sensor is used for recording measurement values on body fluids or excretions from the person (e.g., for determining a concentration of a substance) and is used for this purpose at a given time and then “put aside”.

Preferably, multiple sensors which capture multiple physiological parameters in parallel are present in a single device (sensor device). For example, commercially available fitness trackers and smart watches are capable of measuring heart rate, step count (using a pedometer) and acceleration (using an acceleration sensor) in parallel. Preferably, such a sensor device is usable in a transportable and mobile manner and implemented such that it is continuously carried during the observation period by the person to be monitored.

Preferably, a sensor measures one or more physiological parameters continuously. Generally, an individual measurement requires a certain time span. The term “continuously” means that the sensor carries out a multiplicity of individual measurements over an examination period which generally extends over several hours to days or weeks, with the time interval between two successive individual measurements being sufficiently small for there to be a detectable temporal development of the variable measured (in comparison with larger time intervals, which merely show snapshots, but in which it is not possible to draw any conclusions about the temporal profile).

The measurement values captured by the sensor are analyzed by a monitoring unit for signs of the presence of an adverse event.

An “adverse event” is any disadvantageous medical incident in a person, especially after ingestion of a drug and/or after the use of a medical device and/or as part of some other medical treatment. With said incident, there need not necessarily be a causal relationship with the treatment. Therefore, an adverse event can be an unfavorable and unintended sign (e.g., a striking laboratory result), symptom or a disease in which there is, in each case, a temporal link with, for example, the use of a drug, irrespective of whether a link with said drug is assumed. The term also covers laboratory results or events from other diagnostic measures considered clinically relevant (e.g., which require unplanned diagnostic methods or treatments or which result in an exit from the study).

An adverse event can, for example, be:

a (new) disease,

a deterioration in a sign or symptom of an accompanying disease,

an effect of the study medication,

an effect of a comparative product,

an event when a drug is used beyond the scope of authorization, occupational exposure, lack of drug effect (including unusual nonefficacy), medication error, overdosage, abuse of a drug, misuse of a drug or drug dependency per se and also any event arising therefrom,

an effect with regard to an improvement in a preexisting disease (unexpected therapeutic use is observed),

product exposure via mother/father (exposure during conception, pregnancy, birth and breastfeeding).

The measurement values are analyzed by the monitoring unit. The goal of the analysis is to identify deviations from defined target values in the measurement values. A deviation of the measurement values from defined target values indicates an adverse event.

The target values are generally predefined, i.e., defined before the start of the medical treatment. The (pre)defined values can, for example, be values which occur in a healthy person (standard values). This shall be elucidated using two examples. The usual body-core temperature in a person is between 36.3° C. and 37.4° C. All temperatures in the range between 36.3° C. and 37.4° C. are defined as target values. A sensor captures body-core temperature as a physiological parameter in a person and measures values above 37.4° C. This is a deviation from the defined target values; an adverse event is present. The resting heart rate in a healthy person is usually 60 to 100 beats per minute. Heart rates in the range from 60 to 100 beats per minute are defined as target values. A sensor captures heart rate as a physiological parameter in a person and measures values above 100 beats. This is a deviation from the defined target values; an adverse event is present.

It is also conceivable to not use the standard values from a healthy person as reference for the target values, but to define the target values individually for the specific medical treatment and/or for the person to be specifically monitored. It is conceivable that the target values are specified (defined) on the basis of the individual health (or disease) status of the person to be monitored. It is also conceivable that the target values are automatically specified on the basis of personal data from the person to be monitored. For example, it is conceivable that, on the basis of personal data for each person, an expert system individually specifies those target values, the exceedance and/or undershooting of which of is to be regarded as a sign of an adverse event. The expert system can have been created beforehand on the basis of interviews with human experts.

The analysis of the measurement values captured by the sensor and the identification of deviations in the measurement values from defined target values are done by a monitoring unit. Sensor and monitoring unit can be components of a single device; however, they can also be components of different devices.

According to some embodiments, a control unit which receives measurement values from the sensor and transmits them to the monitoring unit for analysis is present. Control unit and monitoring unit can be components of a single device; however, they can also be components of different devices.

The monitoring unit is configured such that it examines the measurement values for deviations from defined target values. Said deviations can be values above a defined limit and/or below a defined limit.

If the monitoring unit detects a deviation from a defined target value, the monitoring unit can transmit a signal to the control unit. The signal indicates that a deviation of the measurement values from a defined target value has been observed within an observation period—an adverse event has thus occurred within the observation period.

The control unit is configured such that, in the event of a signal for the presence of an adverse event that has been transmitted by the monitoring unit, it transmits the measurement values to a classification unit.

The classification unit and the control unit can be components of a single device;

however, they can also be components of different devices.

Usually, what can be transmitted includes the measurement values of the observation period in which the deviations from the defined target values occurred.

Preferably, what can be transmitted includes measurement values which are temporally immediately before and/or immediately after said observation period.

In addition to the measurement values, further personal data and/or environmental data can be ascertained and forwarded (usually by the control unit) to the classification unit.

The personal data and/or environmental data can, for example, be captured by means of one or more further sensors and/or read from one or more databases.

The personal data and/or environmental data serve to identify the cause of the adverse event observed. The personal data and/or environmental data characterize the state of the person and/or the environmental conditions to which the person has been exposed and which may have an influence on the physiological parameters monitored. The personal data and/or environmental data can be used to indicate or rule out the medical treatment as the cause of the adverse event.

Examples of suitable personal data are:
weight of the person
age of the person
sex of the person
activity level
body temperature
time and quantity of an ingested drug
time and quantity of foodstuffs such as, for example, alcohol, coffee and others
blood pressure values
heart rate
respiratory rate
falls
fatigue level (e.g., via face recognition or other methods)
stress level (e.g., via voice recognition)
pain level (e.g., via face recognition or other methods)
blood sugar level
bilirubin level (e.g., via optical sensor) and much more.

Examples of suitable environmental data are:
weather (air pressure, air humidity, temperature, solar radiation and the like)
location data (geographic location) of the person
speed of the person
air composition (especially oxygen concentration)
exposure of the person to substances which have an influence on the health status
time and date at which the potentially adverse event occurred

and much more.

The classification unit is configured such that, on the basis of the measurement values having deviations from defined target values and the personal data and/or environmental data, it performs a classification of the observation period in which the deviations were observed and an adverse event thus occurred.

The observation period in which an adverse event occurred is assigned to one of at least three classes:

Class A: the deviations from defined target values are not a result of the medical treatment.

Class B: the deviations from defined target values are a result of the medical treatment.

Class C: a clear statement about the cause of the deviations from defined target values cannot be made.

It is also conceivable to perform a classification into more than three classes. For example, a distinction could be made between an adverse event and a severe adverse event.

An adverse event is severe when it:

results in death,

is life-threatening,

requires a hospital stay as inpatient or the prolonging of an existing hospital stay,

results in a permanent or considerable disability or impairment,

concerns a congenital malformation or a birth defect, or

is medically important.

Classification is based on the time period (the “event”) in which signs of the presence of an adverse event occurred. Classification is about classifying the time period/the event more exactly and reducing the number of checks to be carried out by a human expert to determine whether the adverse event is attributable to the medical treatment, by separating cases for which the medical treatment can be identified as the cause of the adverse event and cases for which a cause of the adverse event other than the medical treatment can be identified from the cases in which no statement can be made as to whether the adverse event is attributable to the medical treatment or not because of the available database. Only the last-mentioned cases, in which the cause cannot be automatically ascertained on the basis of the available data, must be checked by a human expert.

The following example is intended to illustrate classification. It is conceivable that a person wears a sensor for monitoring heart rate. The sensor measures heart rate as a physiological parameter and transmits the measurement values to a control unit. The control unit forwards the measurement values to a monitoring unit. The monitoring unit identifies measurement values above 100 beats per minute. According to the configuration of the monitoring unit, such a measurement value is a deviation from defined target values. An adverse event is thus present. The monitoring unit transmits a signal to the control unit. The control unit transmits the measurement values to the classification unit. Moreover, the person to be monitored wears an activity tracker. Said activity tracker comprises a pedometer and an acceleration sensor. The control unit is configured such that it receives the measurement data of the pedometer and of the acceleration sensor from the activity tracker. Furthermore, the control unit is configured such that it transmits to the classification unit those measurement data of the pedometer and of the acceleration sensor that were captured in the same period as the measurement values having the deviations. The classification unit is configured such that it analyzes all the transmitted data and performs a classification. The classification unit evaluates all the data in order to decide, on the basis of the data, whether the adverse event (heart rate above 100 beats per minute) can be attributed to a medical treatment (e.g., the administration of a drug) or whether the adverse event can be attributed to another cause such as, for example, a physical exertion indicated by the pedometer and the acceleration sensor or whether the available database is insufficient for making a statement concerning the causality between adverse event and medical treatment.

In a preferred embodiment, a self-learning system which is trained before use forms the basis of the classification unit. For example, the training could consist in presenting data sets (measurement values having deviations from defined target values and personal data and/or environmental data) to a human expert. The expert carries out a classification and transfers the classification to the self-learning system, which as a result learns what combination of data leads to what class.

The self-learning system can, for example, be an artificial neural network.

The classification unit is configured such that it transmits the result of the classification to the control unit.

The control unit is configured such that, in the case of a classification into classes A and B, it prompts a transmission unit to transmit a message about the presence of an adverse event to a computer system for capturing adverse events. Said computer system is usually a computer system to which the control unit can connect via a network. In addition to the adverse event, information concerning whether the event to the medical treatment or to another cause and, where appropriate, information concerning the corresponding cause is saved in a database of the computer system.

The control unit is configured such that, in the case of a classification into class C, it prompts a transmission unit to transmit measurement values to a human expert for further clarification. Said human expert can then perform a further check.

### BRIEF DESCRIPTION OF THE DRAWINGS

Exemplary embodiments will be described in the following with reference to the following drawings:

FIG. 1 shows schematically an example of the system according to some embodiments of the invention.

FIG. 2 shows schematically an example of the system according to some embodiments of the invention.

FIG. 3 shows schematically an example of the system according to some embodiments of the invention.

FIG. 4 shows schematically an example of the system according to some embodiments of the invention.

FIG. 5 shows schematically an example of the system according to some embodiments of the invention.

FIG. 6 shows schematically an example of the system according to some embodiments of the invention.

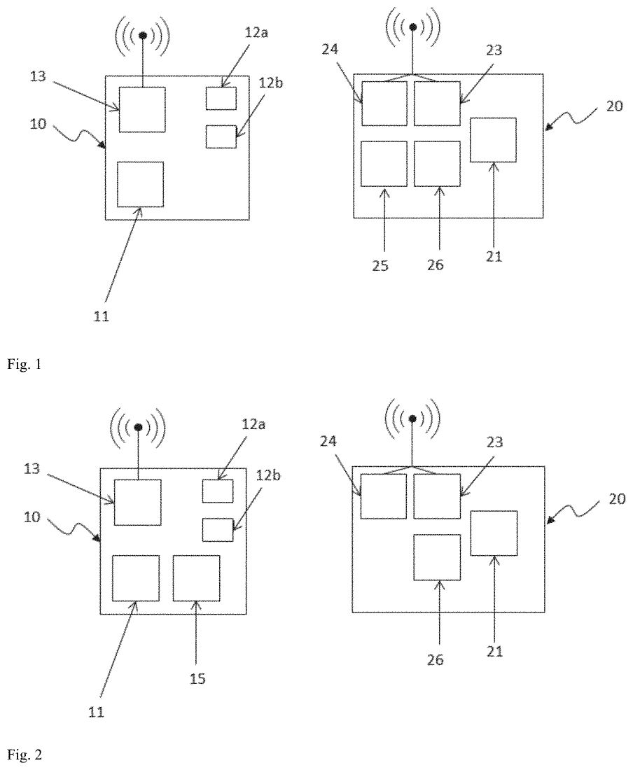

The same reference signs in FIGS. 1, 2, 3, 4, 5 and 6 represent the same components.

### DETAILED DESCRIPTION OF EXEMPLARY EMBODIMENTS

The system according to some embodiments of the invention can have different configurations. In the exemplary embodiment shown in FIG. 1, the system comprises two separate appliances, a sensor device (10) and an evaluation device (20). The sensor device (10) comprises two sensors (12a, 12b) for capturing measurement values of physiological parameters of a person, a transmission unit (13) which can transfer measurement values to the evaluation device (20), and a control unit (11) for controlling the capturing of measurement values and the transmission of measurement values. The evaluation device (20) comprises a receiving unit (24) for receiving measurement values from the sensor unit (10), a monitoring unit (25) for analyzing the received measurement values and for identifying signs of the presence of an adverse event. The evaluation unit (20) furthermore comprises a classification unit (26) for classifying events, a transmission unit (23) for transmitting data, and a control unit (21) for controlling the receiving of measurement values via the receiving unit (24), for controlling the transmission of data by means of the transmission unit and for controlling the components of the evaluation unit (20) and the data flows and signal flows between these components. In the present example, the sensor device (10) captures measurement values of at least two physiological parameters by means of the two sensors (12a, 12b) and transmits measurement values to the evaluation device (20) via a wireless connection (e.g., via a Bluetooth connection). The evaluation device (20) receives measurement values and the monitoring unit (25) analyzes the received measurement values. If the monitoring unit (25) identifies deviations from defined target values in the measurement values, the measurement values and also further personal data and/or environmental data are supplied to the classification unit (26). The classification unit (26) carries out a classification. The control unit (21) initiates various actions depending on the result of the classification: in the case of a class A or a class B, the control unit (21) prompts the transmission of a message about the presence of an adverse event to a computer system for capturing adverse events by means of the transmission unit (23); in the case of a class C, the control unit (21) prompts the transmission of the measurement values to an expert for further clarification by means of the transmission unit (23).

FIG. 2 shows an example of the system according to some embodiments of the invention. The exemplary system shown in FIG. 2 comprises two separate devices (appliances): a sensor device (10) and an evaluation device (20). The evaluation device (20) does not comprise a monitoring unit. Instead, a monitoring unit (15) is part of the sensor device (10). Measurement values which can be captured by one of the two sensors (12a or 12b) or by both sensors (12a and 12b) can be analyzed by the monitoring unit (15) for signs of the presence of an adverse event. If such a sign is identified, the control unit (11) prompts a transmission of the measurement values by means of the transmission unit (13) to the receiving unit (24) of the evaluation device (20). The control unit (21) of the evaluation device (20) prompts a closer analysis of the transmitted measurement values and a classification of the event by means of the classification unit (26), possibly with use of further data.

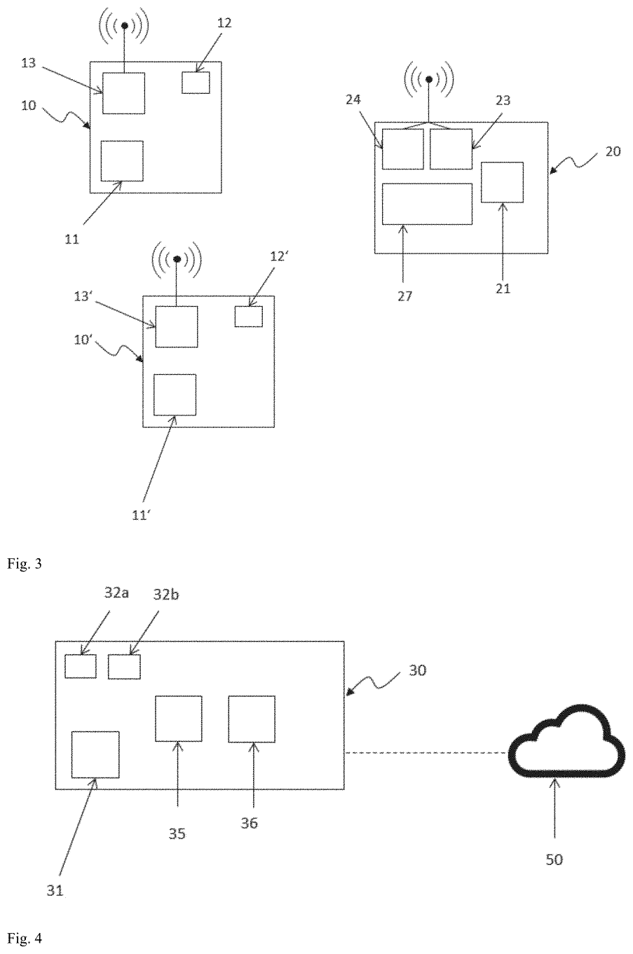

FIG. 3 shows an example of the system according to some embodiments of the invention. The exemplary system shown in FIG. 3 comprises three separate devices (appliances): a first sensor device (10), a second sensor device (10′) and an evaluation device (20). Each of the sensor devices (10, 10′) comprises a sensor (12, 12′), a control unit (11, 11′) and a transmission unit (13, 13′). Both sensor devices (10, 10′) can be configured such that they measure values of one or more physiological parameters and transmit the measurement values to the evaluation unit (20). The evaluation unit (20) receives measurement values and forwards them to a combined monitoring and classification unit (27) for further analysis. The combined monitoring and classification unit (27) can be configured such that it detects signs of the presence of an adverse event in measurement values and classifies the event underlying the measurement values, possibly with use of further data.

FIG. 4 shows an example of the system according to some embodiments of the invention. The exemplary system shown in FIG. 4 comprises a single device (appliance): a combined sensor and evaluation device (30). The combined sensor and evaluation device (30) comprises two sensors (32a, 32b), a control unit (31), a monitoring unit (35) and a classification unit (36). The combined sensor and evaluation device (30) can be connected to one or more further computer systems (depicted by a cloud 50) via a network (depicted by the dashed line). In the case of a classification into class A or class B, the control unit (31) prompts the transmission of a message about the presence of an adverse event to a computer system via the network. In the case of a classification into class C, the control unit (31) prompts the transmission of measurement values having signs of the presence of an adverse event to an expert for further clarification via the network.

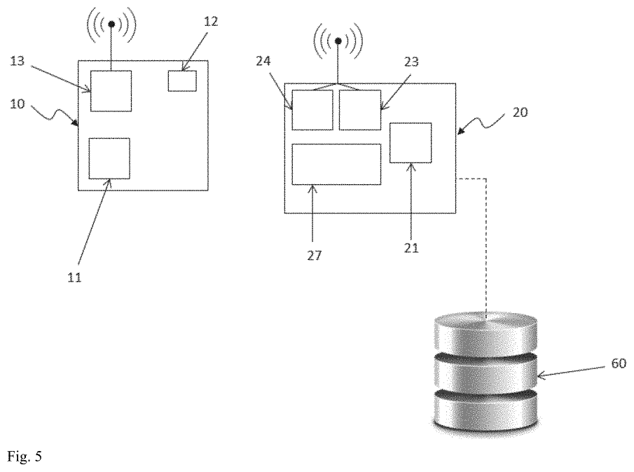

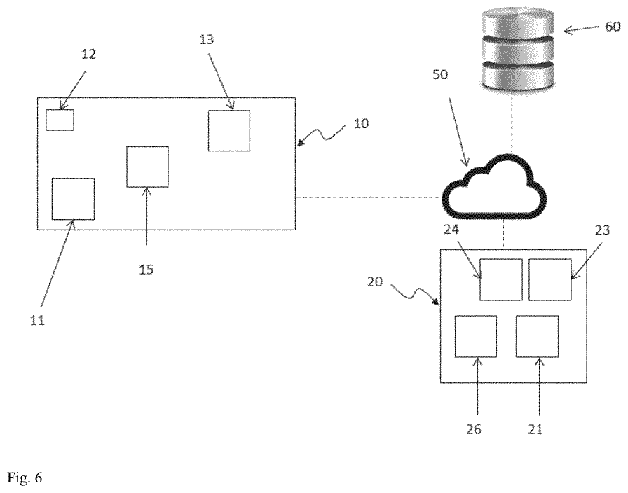

FIG. 5 shows an example of the system according to some embodiments of the invention. The exemplary system shown in FIG. 5 comprises two separate devices (appliances): a sensor device (10) and an evaluation device (20). The evaluation device (20) can be connected to a database (60) via a network (depicted by the dashed line). The classification by means of the combined monitoring and classification unit (27) can be done using further data relating to the person, who is monitored by means of the sensor (12), the further data being obtained from the database (60).

FIG. 6 shows an example of the system according to some embodiments of the invention. The exemplary system shown in FIG. 6 comprises two separate devices (appliances): a sensor device (10) and an evaluation device (20). The sensor device (10) comprises a control unit (11), a sensor for capturing measurement values, a monitoring unit (15) for identifying signs of an adverse event in the measurement values, and a transmission unit (13). The control unit can be configured such that it transmits measurement values to the evaluation device (20) via the transmission unit (13) and via a network (50) if the monitoring unit (15) has identified a sign of the presence of an adverse event in the measurement values. The evaluation device comprises a receiving unit (24), a control unit (21), a classification unit (26) and a transmission unit (24). The classification unit can use further data, which it retrieves from a database (60) via the network (50), for the classification.

For all the embodiments shown here that comprise more than one sensor, it is conceivable that only the measurement values of one of the sensors are examined for a sign of the presence of an adverse event. The measurement values of whichever is the other sensor can, for example, be used as personal data and/or environmental data for the classification.

1. A method for automatically detecting, capturing and processing adverse events as part of a clinical study, a noninterventional study or a therapy, the method comprising:
capturing, using one or more sensors, measurement values of one or more physiological parameters in a person who is being subjected to a medical treatment as part of the study or the therapy;
analyzing the measurement values and identifying deviations from defined target values in the measurement values within an observation period;
ascertaining further personal data and/or environmental data;
classifying the observation period on the basis of the deviations from defined target values and the personal data and/or environmental data into one of:
class A: the deviations from defined target values are not a result of the medical treatment,
class B: the deviations from defined target values are a result of the medical treatment,
class C: a clear statement about the cause of the deviations from defined target values cannot be made; and
transmitting a message about the presence of an adverse event to a computer system for capturing adverse events in response to classifying the observation period into class A or class B, or transmitting measurement values to an expert for further clarification in response to classifying the observation period into class C.

2. The method of claim 1, wherein personal data and/or environmental data which were ascertained by one or more further sensors and/or read from one or more databases are used for the classification.

3. The method claim 1, wherein the one or more sensors are configured to capture measurement values of one or more of the following physiological parameters: body weight, body temperature, heart rate, heart rhythm, blood pressure, skin conductance, tremor (frequency), electrolyte/protein concentration or composition in body fluids, activity of specific brain areas, electrical activities of cardiac muscle fibers, central venous pressure, arterial oxygen saturation, respiratory rate.

4. The method of claim 1, wherein one or more of the following personal data and/or environmental data are used for the classification: weight of the person, age of the person, sex of the person, time and/or date at which the potentially adverse event occurred, activity level, body temperature, time and quantity of an ingested drug, time and quantity of ingested foodstuffs, blood pressure values, heart rate, respiratory rate, time and severity of a fall, location data of the person, speed of the person, fatigue level of the person, stress level of the person, pain level of the person, blood sugar level of the person, bilirubin level of the person, weather data at the time of the potentially adverse event.

5. The method of claim 1, wherein the medical treatment involves administering a drug to the person and the classification serves to identify or rule out the drug as the cause of an observed adverse event.

6. The method of claim 1, wherein the computer system for capturing adverse events includes a case report form in which the adverse events occurring as part of the study or the therapy and the causal links thereof with the medical treatment are to be documented.

7. The method of claim 1, wherein the one or more physiological parameters comprises a heart rate of the person who is being subjected to medical treatment, wherein the method further comprises ascertaining measurement values relating to an activity of the person within the observation period, and wherein class A includes that the deviations from defined target values are a result of the activity of the person.

8. A system for automatically detecting, capturing and processing adverse events as part of a clinical study, a noninterventional study or a therapy, comprising:
one or more sensors configured to automatically capture measurement values of one or more physiological parameters in a person who is being subjected to a medical treatment; and
one or more processors configured to:
analyze the measurement values,
identify deviations from defined target values in the measurement values,
analyze the measurement values having deviations from defined target values and further personal data and/or environmental data, and
perform a classification into one of:
class A: the deviations from defined target values are not a result of the medical treatment,
class B: the deviations from defined target values are a result of the medical treatment,
class C: a clear statement about the cause of the deviations from defined target values cannot be made; and
wherein in accordance with classification into class A or class B, the one or more processors are configured to transmit a message about the presence of an adverse event to a computer system for capturing adverse events, and in accordance with classification into class C, the one or more processors are configured to to transmit measurement values to an expert for further clarification.

9. The system of claim 8, comprising a sensor device, said sensor device comprising the one or more sensors for the automatic capturing of measurement values of one or more physiological parameters in the person, wherein the sensor device is a mobile, wearable sensor configured to be permanently carried by the person over a monitoring period of at least one day and preferably of at least one week.

10. The system of claim 8, comprising a sensor device, said sensor device comprising the one or more sensors for the automatic capturing of measurement values of one or more of the following physiological parameters: body weight, body temperature, heart rate, heart rhythm, blood pressure, skin conductance, tremor (frequency), electrolyte/protein concentration or composition in body fluids, activity of specific brain areas, electrical activities of cardiac muscle fibers, central venous pressure, arterial oxygen saturation, respiratory rate.

11. The system of claim 8, wherein the one or more processors are configured to use one or more of the following personal data and/or environmental data for the classification: weight of the person, age of the person, sex of the person, time and/or date at which the potentially adverse event occurred, activity level, body temperature, time and quantity of an ingested drug, time and quantity of ingested foodstuffs, blood pressure values, heart rate, respiratory rate, time and severity of a fall, location data of the person, speed of the person, fatigue level of the person, stress level of the person, pain level of the person, blood sugar level of the person, bilirubin level of the person, and weather data at the time of the potentially adverse event.

12. The system of claim 11, wherein the system is configured to read personal data and/or environmental data from one or more databases and use the personal data and/or environmental data for the classification.

13. The system of claim 8, wherein a self-learning system, preferably an artificial neural network, forms the basis of the classification.

14. The system of claim 8, comprising
a first sensor for monitoring a heart rate of the person,
a second sensor for measuring an activity level of the person,
an electronic case report form for documenting adverse events occurring as part of the study or therapy and causal links of the adverse events with the medical treatment,
wherein the one or more processors of the system are further configured to:
analyze measurement values relating to heart rate and identify deviations from defined target values in the measurement values relating to heart rate,
analyze measurement values relating to the activity-level of the person, and
transmit a message about the presence of an adverse event to the electronic case report form in response to classification into class A or class B, and
wherein class A includes that the deviations from defined target values are a result of the activity of the person.

15. A non-transitory computer readable program comprising instructions that, when executed by the one or more processors, cause the one or more processors to:
receive measurement values of one or more physiological parameters of a person who is being subjected to a medical treatment from a sensor,
analyze the measurement values and identify deviations from defined target values in the measurement values within an observation period,
classify the observation period on the basis of the deviations from defined target values and personal data and/or environmental data into one of:
class A: the deviations from defined target values are not a result of the medical treatment,
class B: the deviations from defined target values are a result of the medical treatment,
class C: a clear statement about the cause of the deviations from defined target values cannot be made, and
transmit a message about the presence of an adverse event to a computer system for capturing adverse events in response to classification into classes A or B, or transmit measurement values to an expert for further clarification in response to classification into class C.
